(19) United States
(12) Patent Application Publication
(10) Pub. No.: US 2023/0381546 A1
(43) Pub. Date: Nov. 30, 2023

(54) Non-Cavitational Mechanical Pulsed Ultrasound Therapy

(71) Applicant: Wisconsin Alumni Research Foundation, Madison, WI (US)

(72) Inventor: John-Paul Yu, Madison, WI (US)

(21) Appl. No.: 18/200,869

(22) Filed: May 23, 2023

Related U.S. Application Data
(63) Continuation of application No. 63/346,074, filed on May 26, 2022.

(51) Int. Cl. A61N 7/02; A61B 17/00

(52) U.S. Cl. CPC A61N 7/02; A61B 17/00; A61N 2007/025; A61B 2017/00154; A61N 2007/0004

(57) ABSTRACT

Short ultrasound pulses or bursts, delivered at intermediate intensity to the target tissue transmits acoustic radiation forces into the targeted tissue without generating acoustic cavitation and without cellular fractionization. Thus, the present invention exploits the mechanical effects of high-intensity pressure fields only, without cavitation, by optimizing the interaction between acoustic pulses and radiation forces to apply mechanical forces to tissue to generate a broad spectrum of desired and varied bioeffects.

### CROSS-REFERENCE TO RELATED APPLICATIONS

This application claims the benefit of U.S. Provisional Patent Application No. 63/346,074, filed May 26, 2022, which is incorporated by reference herein in its entirety.

### STATEMENT REGARDING FEDERALLY SPONSORED RESEARCH OR DEVELOPMENT

--

### BACKGROUND OF THE INVENTION

The present invention relates to focused ultrasound (FUS) techniques focused on target volumes inside the body in a manner which induces tissue disturbance and more particularly to focused ultrasound techniques that alter tissue microenvironments without ablation, cavitation, or heating effects.

The development of high-intensity focused ultrasound (HIFU) to noninvasively treat disease is part of a comprehensive drive towards less invasive yet effective treatments. With focused ultrasound, an acoustic lens is used to concentrate multiple intersecting beams of ultrasound on a target deep in the body with extreme precision and accuracy.

Histotripsy is a focused ultrasound technique which applies acoustic energy generated by an extracorporeal ultrasound transducer focused on a target volume within the body. Histotripsy exploits the mechanical effects of high-intensity pressure fields to impart mechanical stresses and strains on the cells and tissues of the target volume producing cellular and tissue disruption, i.e., tissue ablation, but with a very low duty cycle to minimize tissue heating.

Specifically, histotripsy optimizes the interaction between acoustic pulses and gas filled microbubbles, i.e., cavitation nuclei, within the human tissue. The process employs high intensity sound waves and short ultrasound (US) pulses or bursts to initiate acoustic cavitation. Acoustic cavitation occurs when a sufficiently negative pressure is applied to the tissue, and specifically, is applied to the endogenous gas present in the tissue to cause microbubble formation from fluid vaporization and release of dissolved gas. Once formed, the microbubbles exhibit highly dynamic patterns of oscillation and inertial collapse. The mechanical action of rapid expansion and collapse of dense clouds of microbubbles or “cavitation clouds” impart severe strains and stresses repeated over many US pulses to disrupt the cells and tissues immediately surrounding the cavitation bubbles. The cavitation clouds are thus controllable to accurately destroy the cells and tissues in the target volume, rendering the tissue into acellular debris. The acellular debris of the target region is then absorbed into the body, for example, within 1 to 2 months, and ablating the tissue. The duty cycle of the US pulses is kept very low (e.g., ≤4% and sometimes ≤2% and sometimes ≤1%) to minimize tissue heating, thus, giving the tissue time to cool between pulses preventing thermal damage.

Referring to the US pulse seen in FIG. 1, typical histotripsy parameters employ extremely high pressure waves with high pressure amplitudes (e.g., peak positive pressure (P+) of at least 50 MPa and peak negative pressure (P−) of at least 15 MPa) to the tissue to generate acoustic cavitation which occurs when the negative pressure threshold is reached within the tissue (e.g., less than 28 MPa for most tissues). Thus, the “cavitation threshold” is the minimum negative pressure amplitude at which pre-existing microbubbles in the tissue begin to oscillate or collapse.

Typical histotripsy parameters (e.g., for intrinsic threshold, shock-scattering, boiling type histotripsy procedures) used to reach the cavitation threshold compared to HIFU parameters are summarized in the table of FIG. 2.

Histotripsy provides several advantages to conventional thermal ablation methods such as HIFU because it is non-thermal, non-invasive, and provides high precision, real-time monitoring/feedback and tissue liquefaction. For example, using histotripsy, tissue ablation can be confined to highly precise target volumes guided by real-time ultrasound monitoring of treatment progression. The histotripsy process can be used to isolate cavitation mechanical effects and minimize tissue heating effects. The result is fractionated tissue that appears hypoechoic as many of the structures that would have reflected ultrasound energy have been broken down and homogenized.

Current clinical applications for histotripsy include treatments and therapies for breast cancer, prostate cancer, several cardiac applications, and various benign diseases including prostatic benign prostatic hyperplasia (BPH) and breast fibroadenoma.

Methods of using pulsed cavitational ultrasound therapy are described in U.S. 2007/0083120 and U.S. 2010/0069797, both entitled, “Pulsed cavitational ultrasound therapy,” and hereby incorporated by reference.

### SUMMARY OF THE INVENTION

The present inventor has found that employing short ultrasound pulses or bursts, but at a lower, intermediate intensity to the target tissue (compared to high-intensity pulses delivered during histotripsy) will cause acoustic radiation forces to be transmitted into the target tissue without generating acoustic cavitation and without cellular fractionization. Thus, the present invention exploits the mechanical effects of high-intensity pressure fields without cavitation and without heating by optimizing the interaction between acoustic pulses and radiation forces to apply a negative pressure to the tissue but that does not reach the negative pressure threshold necessary to cause cavitation encountered in histotripsy.

Thus, the acoustic pulses of the present invention provide smaller pressure waves (i.e., lower peak positive and negative pressure amplitudes) while still producing precise and accurate lesions of disturbed tissue with sharp boundaries between treated and untreated tissue.

Further, the narrow width of each ultrasound pulse highly restricts the degree to which bone aberrations, such as from ribs or skull, can induce and distort the ultrasound focus and the short pulse duration allows the ultrasound pulses to be delivered rapidly while maintaining low duty cycles to avoid complications of tissue heating that are often encountered in other FUS technologies such as HIFU.

The present invention provides pulsed ultrasound therapy for mechanical tissue shaping (i.e., the “histoplasty” process) coupled with real-time monitoring/feedback which provides tissue disturbance (i.e., shear forces, compressive forces, high pressure, chemical effects, conformational effects) without cavitation and cellular fractionization while minimizing known limitations such as tissue heating, ultrasound aberration, and tissue inflammation that are associated with other microbubble-based focused US approaches such as histotripsy.

The mechanical effects of the tissue disturbance without cellular fractionization can achieve a wide range of non-ablative soft tissue bioeffects depending on the targeted tissue and the peak pressure amplitudes use.

The present invention provides a method for controlled mechanical deformation of soft tissue having a cavitation threshold initiating a bubble cloud, comprising outputting a treatment ultrasound pulse sequence at a treatment portion of the soft tissue without initiation of a bubble cloud in said treatment portion of the soft tissue in response to the treatment ultrasound pulse sequence; wherein the treatment ultrasound pulse sequence is at a pulse intensity that is less than the cavitation threshold and at least partially deforms the soft tissue in the treatment portion without initiating a bubble cloud.

It is thus a feature of at least one embodiment of the invention to provide mechanical tissue effects without tissue ablation and without tissue heating.

A peak negative pressure of the treatment ultrasound pulse sequence may be less than or equal to 20 MPa. A peak negative pressure of the treatment ultrasound pulse sequence may be less than or equal to 15 MPa. A peak negative pressure of the treatment ultrasound pulse sequence may be less than or equal to 10 MPa.

A peak positive pressure of the treatment ultrasound pulse sequence may be less than MPa.

A frequency of the treatment ultrasound pulse sequence may be between 500 kHz and 1 MHz.

A pulse repetition frequency (PRF) of the treatment ultrasound pulse sequence may be between 250 Hz and 750 Hz.

A duty cycle of the treatment ultrasound pulse sequence may be less than or equal to 10%. A duty cycle of the treatment ultrasound pulse sequence may be less than or equal to 2%. A duty cycle of the treatment ultrasound pulse sequence may be less than or equal to 1%.

The pulse length of the treatment pulse may be less than or equal to 15 μsec. A pulse length of the treatment pulse may be less than or equal to 20 μsec.

A number of pulses in the treatment ultrasound pulse sequence may be 10 to 50 pulses.

An exposure duration of the treatment ultrasound pulse sequence may be less than or equal to 60 sec. An exposure duration of the treatment ultrasound pulse sequence may be less than or equal to 30 sec. An exposure duration of the treatment ultrasound pulse sequence may be less than or equal to 10 sec.

The treatment ultrasound pulse sequence may be applied to at least one of a breast cancer tissue, liver cancer tissue, and pancreas cancer tissue.

The method may further comprise monitoring an amount of deformation of the soft tissue in the treatment portion.

The method may further comprise adjusting the treatment ultrasound pulse sequence based on the amount of deformation of the soft tissue in the treatment portion.

The present invention also provides a method for controlled mechanical disruption of fibrotic extracellular matrix in a human tissue having a cavitation threshold initiating a bubble cloud in the fibrotic extracellular matrix, comprising outputting a treatment ultrasound pulse sequence at a treatment portion of the fibrotic extracellular matrix without initiation of a bubble cloud in said treatment portion of the fibrotic extracellular matrix in response to the treatment ultrasound pulse sequence; wherein the treatment ultrasound pulse sequence is at a pulse intensity that is less than the cavitation threshold and at least partially deforms the fibrotic extracellular matrix without initiating a bubble cloud in the fibrotic extracellular matrix.

The fibrotic extracellular matrix is of at least one of a breast cancer tissue, liver cancer tissue, and pancreas cancer tissue.

The method may further comprise monitoring an amount of fibrotic extracellular matrix disruption and adjusting the treatment ultrasound pulse sequence based on the amount of disruption in the treatment portion.

The method may further comprise monitoring a collagen density in the fibrotic extracellular matrix and adjusting the treatment ultrasound pulse sequence based on the collagen density in the fibrotic extracellular matrix in the treatment portion.

The method may further comprise monitoring an amount of penetration of therapeutics into the human tissue and adjusting the treatment ultrasound pulse sequence based on the amount of penetration of therapeutics into the human tissue in the treatment portion.

These particular objects and advantages may apply to only some embodiments falling within the claims and thus do not define the scope of the invention.

### BRIEF DESCRIPTION OF THE DRAWINGS

FIG. 1 is an exemplary graph showing prior art waveforms of the ultrasound pulses delivered during histotripsy treatments;

FIG. 2 is a table showing typical histotripsy parameters (e.g., for intrinsic threshold, shock-scattering, boiling type histotripsy procedures) used to reach the cavitation threshold compared to HIFU parameters;

FIG. 3 is a schematic diagram of a mechanical interaction between ultrasound waves and soft tissue and cells of the patient tissue to exhibit deformation of the tissue through shear stress waves in accordance with the teachings of the present invention;

FIG. 4 is a schematic illustration of an exemplary apparatus for performing pulsed non-thermal, non-cavitational ultrasound therapy constructed in accordance with the teachings of the present invention;

FIG. 5 is an exemplary graph showing waveforms of the ultrasound pulses delivered using the apparatus of FIG. 4 during histoplasty treatments according to the teachings of the present invention;

FIG. 6 is a table showing transducer parameters used in histoplasty according to the teachings of the present invention;

FIG. 7 is a table showing lower peak negative pressures peak negative pressure set to prevent cavitation and with varying transducer frequency and pulse repetition frequency (PRF) according to the teachings of the present invention;

FIG. 8 show ultrasound images of pre-treatment and post-treatment second harmonic generation imaging of a 4T1 breast cancer tissue xenograft demonstrating the decrease in number and size of collagen fibers post-histoplasty;

FIG. 9 is a graph showing how histoplasty increases intratumoral diffusion, penetration, and residence time of therapeutics in a 4T1 murine model of breast carcinoma (mice bearing 4T1 mammary tumors are injected with liposomal doxorubicin (Doxil) and Doxil fluorescence intensity is measured 30 min, 1 day and 2 days following sham or histoplasty treatment showing histoplasty treated mice demonstrate statistically significant increased uptake of Doxil when compared to sham and untreated animals (**, p<0.01));

FIG. 10 are graphs showing tumor flow cytometry showing CD4/CD45 and CD8/CD45 ratios of control and histoplasty treated tumors demonstrate a significant increase in CD8+ T-cells in histoplasty treated tumors (left panel) and a statistically significant increase in dendritic cells is also seen in histoplasty treated tumors (right panel) (*, p<0.05; **, p<0.01); and

FIG. 11 is a table showing histoplasty parameters to be tested in Examples 6 and 7 (Low, intermediate, and high peak negative pressures are tested. Initial duty cycle parameters are chosen to negate any thermal effects. 6 replicates are performed for each combination of peak negative pressure, duty cycle, and duration. For all conditions, fundamental frequency=1 Mhz, PRF=500 Hz).

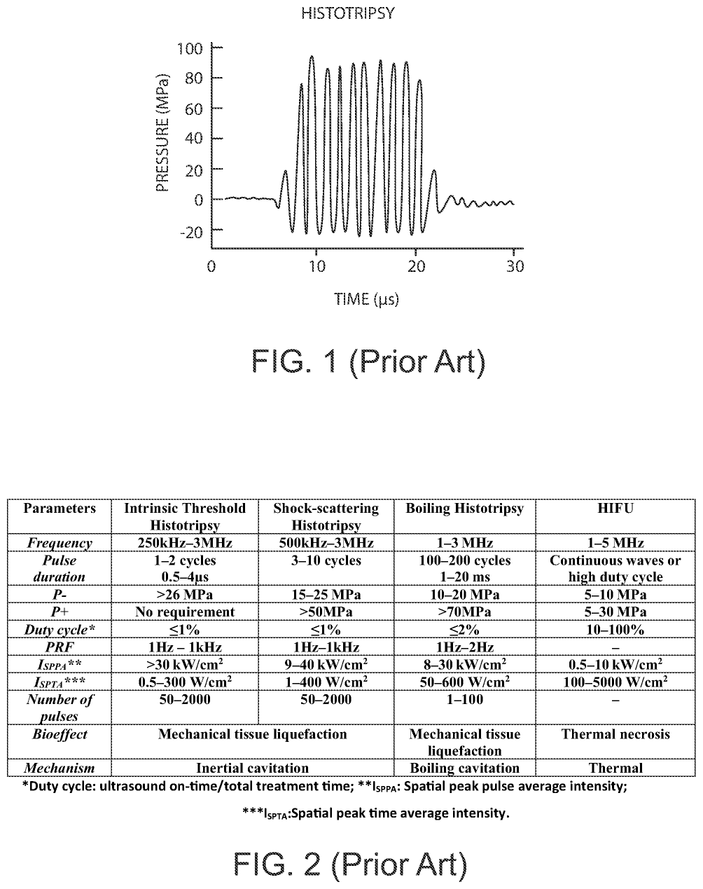

### DETAILED DESCRIPTION OF THE INVENTION

Ultrasound waves interact with soft tissue in numerous ways to yield a range of biophysical effects. Broadly, these bioeffects can be divided into “thermal” and “non-thermal” mechanisms.

“Thermal” bioeffects of FUS have been extensively studied and are harnessed to perform targeted non-invasive thermal ablations throughout the body. For example, in the central nervous system, magnetic resonance-guided FUS has been approved by the U.S. Food and Drug Administration to ablate a single focal volume in the central nervous system as a therapy for essential tremor.

“Non-thermal” bioeffects are mechanical bioeffects present during insonation with FUS. Although the mechanical bioeffects associated with FUS are generally considered ancillary and representative of how ultrasound energy is dissipated in tissue, recent work demonstrates the capacity for FUS to generate clinically salient non-thermal mechanical bioeffects for therapeutic applications without the use of heat. Histotripsy uses pressure waves with high rarefactional (negative) amplitudes to draw dissolved gas out of liquid tissue to form cavitation bubbles, which then undergo inertial cavitation to cause targeted non-thermal and mechanical ablation of tissue. However, beyond tissue ablation, the present invention contemplates that FUS can be used to produce other mechanical bioeffects in soft tissues.

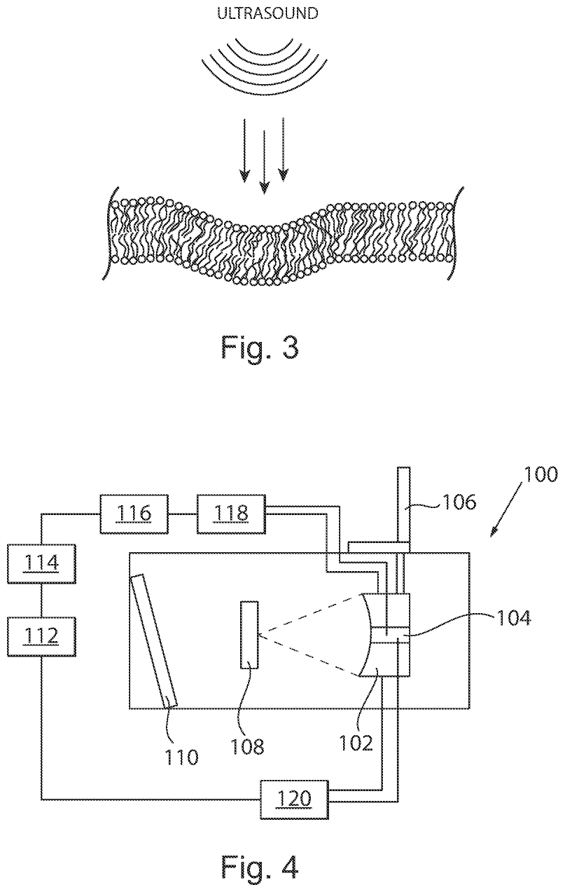

Referring to FIG. 3, the present invention contemplates the use of intermediate-intensity ultrasound directed to the target tissue (i.e., without tissue ablation or bulk tissue fractionization which fractionates portions of a cell or fractionates on a cellular level) to produce mechanical bioeffects in soft tissue such as would cause mechanical deformation, mechanical disruption of the tissue, or cellular or tissue remodeling to change tissue or cellular interactions to produce desired effects without cavitation.

Therefore, the present invention produces mechanical bioeffects without heating and without bubble formation using intermediate amplitude acoustic therapy pulses.

### Focused Ultrasound for Mechanical Tissue Shaping

The present disclosure uses pulsed non-thermal, non-cavitational high intensity focused ultrasound to affect tissue shaping assisted processes such as mechanical deformation, cell or tissue remodeling, and drug delivery, in a predictable and controllable manner for mechanically affecting tissues for therapeutic applications. The pulsed non-thermal, non-cavitational therapy process is similar to histotripsy, in that soft tissues are mechanically disturbed, but does not employ cavitation to induce cellular destruction. The present process of pulsed non-thermal, non-cavitational ultrasound is also referred to herein as histoplasty, connoting essentially the “shaping” of soft tissues. The histoplasty process of the present teachings can, at least in part, involve the mechanical movement of tissue, i.e., stresses and strains, without the creation and maintenance of ensembles of microbubbles and, in some embodiments, the use of feedback in order to optimize the process based on observed stress and strain tissue dynamics in real time.

In the past, cavitation was avoided in therapeutic applications because its results were unpredictable with regards to both location of damage and thresholds for damage production, and therefore, the damage produced was spatially irregular. However, through the invention of the histotripsy process, pulsed ultrasound was used to produce microbubbles, both in the form of contrast agents and/or other active agents infused into the body or bubbles formed from previous ultrasound exposure, to allow for more predictable damage location and thresholds, with much lower incident intensities for damage production, and production of much more spatially regular lesions.

The present invention builds on this existing technology by using pulsed ultrasound in a manner which uses predictable cavitation thresholds to avoid the production of microbubbles but still providing much lower incident intensities for damage production and production of much more spatially regular lesions. In this respect, the problems and limitations of existing US techniques such as HIFU and histotripsy of tissue heating, ultrasound aberration, and tissue inflammation have been improved to produce tissue remodeling instead of tissue destruction or ablation.

Referring to FIG. 4, an exemplary apparatus 100 for performing pulsed non-thermal, non-cavitational ultrasound therapy constructed in accordance with the teachings of the present disclosure is shown. The apparatus can comprise a therapy transducer 102 and a monitoring transducer 104 coupled to a 3-axis positioning system 106. The therapy transducer 102 and monitoring transducer 104 focus ultrasound onto the target tissue 108, backed by a sound absorber 110. Computer control and data collection 112 is coupled to a function generator 114 that is coupled to an amplifier 116 that is coupled to a matching circuit 118 that is coupled to the transducers 102, 104. Computer control and data collection 112 are also coupled to a digital oscilloscope 120, which is further coupled to the transducers 102, 104.

Pulsed non-thermal, non-cavitational ultrasound therapy, or the histoplasty process, according to the present teachings, may include the following steps, namely: therapy step (histoplasty) and feedback step, which are described in further detail below.

During the therapy step, the target volume which is mostly void of micro-nuclei (i.e., small microbubbles) is impinged upon by therapy pulses that produce acute tissue deformation. Each therapy pulse can produce just a small part of the overall therapy effect, which can include mechanical tissue deformation. The overall therapy effect may include applying shear stress and strain on the tissue, applying positive and negative pressure on the tissue, inducing chemical changes in the tissue based on the mechanical deformation, and the like associated with the mechanical forces applied to the tissue.

In one embodiment, the therapy produces the desired therapy effect during the therapy step. In one embodiment, in the therapy step, a series of intermediate intensity pulses are focused into the therapy volume sufficient to produce mechanical deformation but is below a value that would initiate bubble clouds. This intermediate intensity is sufficient to produce adequate mechanical deformation without cavitation. As will be described herein, feedback on the mechanical deformation can be obtained by monitoring the therapy pulse backscatter from the tissue. The backscatter is monitored by the therapy transducer (or subset of therapy transducer array elements) in the receive mode, or by a simple (and separate) monitoring transducer. In some embodiments, multiple transducers can be employed for monitoring feedback.

During the feedback step, the treatment step can be monitored to thereby check overall therapy progression. The feedback and monitoring step allows for various parameters of the pulsed non-thermal, non-cavitational ultrasound process to be varied in real time or in stages, if desired, permitting controlled administration of the ultrasound therapy. For example, the process can be terminated, the extent of therapy measured, and the process restarted. In particular, the feedback step enables adjustment and tuning of the histoplasty process in precise and controlled ways previously unobtainable.

It should be noted that methods of the present teachings can include variations where each of these steps can use different methods of energy delivery with different forms of energy and different feedback schemes. Additional details of various embodiments of each step follow.

Therapy Step:

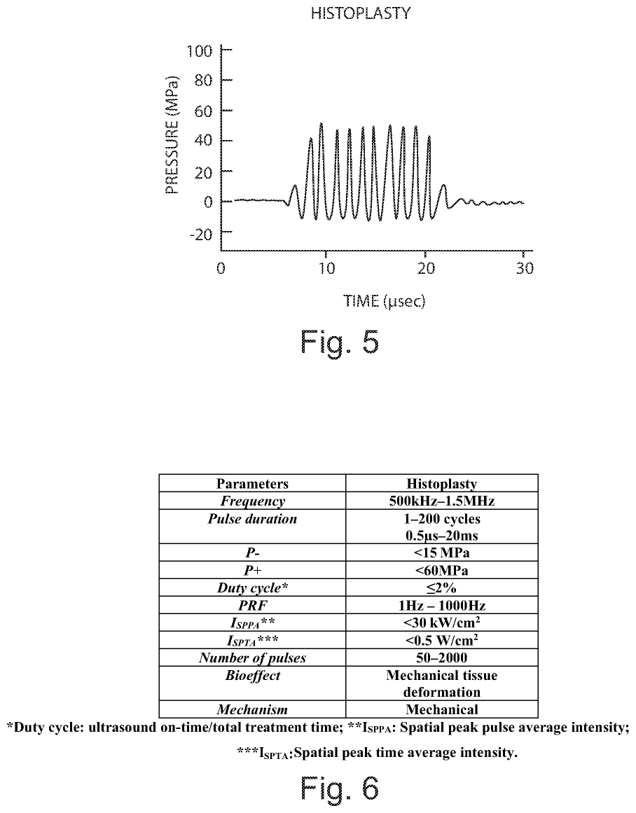

Referring also to FIG. 5, therapy can comprise of a therapy pulse sequence, which is also referred to herein as a therapy sequence, therapy pulse, or therapy. The therapy process is the interaction of ultrasound on therapy tissue to produce tissue deformation (without cavitation and without mechanically subdividing the tissue) within the therapy volume. Therapy energy in the histoplasty process can be acoustic (e.g., ultrasonic).

The transducer or transducers can be either single focus, or multi-focus, or phased arrays where the focus can be scanned in 1, 2, or 3-dimensions. The therapy transducer(s) can be contiguous spatially or can be separated spatially, using multiple windows into the therapy volume. The transducers can also operate at different frequencies individually or as an overall ensemble of therapy transducers. The therapy transducer(s) can also be mechanically scanned to generate larger therapy zones and/or a combination of mechanically and electronically (phased array) scans can be used. The therapy transducer(s) can be intimately involved in the feedback processes and procedures as sources of interrogation sequences or as receivers (or even imagers).

The multiplicity of transducers enables various embodiments where one of the therapy transducers could operate at a significantly lower frequency from the other(s).

In some embodiments, one or more low frequency transducers can act as a “pump” with the other transducer(s) sending pulses (i.e., for therapy or feedback) propagating along with the low frequency pump. For example, if a higher frequency, short therapy pulse arrives in the therapy volume in a particular relation to the phase of the low frequency pump pulse, multiple effects can be obtained therefrom depending on this relative phase relationship. In one embodiment, if the higher frequency pulse arrives at the therapy volume on the peak positive pressure of the pump, the cavitational effect is reduced. Also, if the pump and therapy pulse arrive at different propagation angles, it can serve to spatially sharpen the effective focus of the therapy pulse. The maximum sharpening effect occurs when the pulses arrive having been propagated in opposite directions or 90 degrees from each other.

The therapy transducers (i.e., high and low frequencies) can also operate in conjunction with the feedback transducers to enhance effects. For example, if an imaging transducer is used for feedback, it can be used to enhance the detection of unwanted microbubbles or nuclei. That is, if the imaging pulse arrives in the imaging volume on the rarefactional trough of the pump pulse, any nuclei or microbubbles will have expanded and will be relatively hyperechoic. If the imaging pulse arrives on the peak positive pressure, the nuclei or microbubbles will be smaller in size (compressed) and the image in this interaction zone will be relatively hypoechoic. Thus, by using a difference image, one will see only microbubble activity as the other tissue echoes will be constant (same) in both images.

In some embodiments, the therapy pulse can be used as a pump and the imaging pulse can be propagated therewith. If one or more therapy pulses are focused on a therapy volume or portion of a therapy volume, the intensity can be greater in the focused therapy volume. Therefore, the mechanical effect on tissue will be greater in the focused therapy volume and less away from the focused therapy volume. By co-propagating the imaging and therapy pulse alternately, with the imaging pulse riding on the peak rarefactional pressure of the therapy pulse and the peak positive pressure of the therapy pulse, a difference image will show the greatest difference near the focused therapy pulse(s). The difference will be less away from the focused therapy pulse(s). Thus, this scheme allows direct imaging of the therapy pulse beam pattern. This can be used to identify and locate where the maximum mechanical deformation will occur in the therapy volume before treatment.

Feedback & Monitoring Step:

In some embodiments, feedback enables assessment of parameters related to noninvasive image guided therapy or drug delivery. The methods and devices depend on the fact that the actual therapeutic effect is the progressive mechanical deformation of the tissue that can also provide enhanced drug transport (or other therapeutic or diagnostic effect) over one or more therapy pulses. Thus, the tissues exposed to the histoplasty process are changed physically. These physical changes are much more profound than changes produced by competing therapies. Furthermore, embodiments of the present teachings make it possible to monitor the therapeutic effectiveness both during and after the therapy process.

In some embodiments, feedback and monitoring can include monitoring changes in: speckle reduction in backscatter; backscatter speckle statistics; mechanical properties of tissue (i.e., elastography); shear wave propagation; acoustic emissions, and electrical impedance tomography, as described in more detail below.

Backscatter, Speckle Reduction: Progressively mechanically deformed tissue, in other words disrupted or moved tissue, results in changes in the distribution of acoustic scatter. At some point in the process, the tissue is disrupted or changed in position enough where little ultrasound is scattered, or the amount scattered is reduced significantly. This results in a significant reduction in speckle, which is the coherent constructive and destructive interference patterns of light and dark spots seen on images when coherent sources of illumination are used; in this case, ultrasound. After some treatment time, the speckle reduction may result in a dark area in the therapy volume. Since the amount of speckle reduction is related to the amount of tissue disruption, it can be related to a change in orientation of the tissue and cells. So, treatment can proceed until a desired speckle reduction level has been reached. Speckle is easily seen and evaluated on standard ultrasound imaging systems. Specialized transducers and systems can also be used to evaluate the backscatter changes.

Backscatter, Changes in Speckle Statistics: Speckle in an image persists from frame to frame and changes little as long as the scatter distribution does not change and there is no movement of the imaged object. However, long before the scatters are reduced enough in size to cause speckle reduction, they may be changed sufficiently to be detected by signal processing and other means. This family of techniques can operate as detectors of speckle statistics changes. For example, the size and position of one or more speckles in an image will begin to decorrelate before observable speckle reduction occurs. Speckle decorrelation, after appropriate motion compensation, can be a sensitive measure of the mechanical disruption of the tissues, and thus a measure of therapeutic efficacy. This feedback and monitoring technique permits early observation of changes resulting from the histoplasty process and can identify changes in tissue. For example, this method can be used to monitor the histoplasty process for enhanced drug delivery where tissue is spatially disrupted.

Elastography: As the tissue is further deformed or disrupted, its mechanical properties change from a soft but interconnected solid to a disconnected solid with less cellular interactions. These changes in mechanical properties can be measured by various imaging modalities including MRI and ultrasound imaging systems. For example, an ultrasound pulse can be used to produce a force (i.e., a radiation force) on a localized volume of tissue. The tissue response (displacements, strains, and velocities) can change significantly during histoplasty treatment allowing the state of tissue disruption to be determined by imaging or other quantitative means.

Shear Wave Propagation Changes: The disruption of tissues makes the tissue more “fluid” and less solid and fluid systems generally do not propagate shear waves. Thus, the extent of tissue fluidization (or when cells are connected to less neighboring cells or there are less cellular interactions) provides opportunities for feedback and monitoring of the histoplasty process. For example, ultrasound and MM imaging systems can be used to observe the propagation of shear waves. The extinction of such waves in a treated volume is used as a measure of tissue distortion or disruption. Moreover, dedicated instrumentation can be used to generate and measure the interacting shear waves. For example, two adjacent ultrasound foci might perturb tissue by pushing it in certain ways. If adjacent foci are fluidized, no shear waves propagate to interact with each other. If the tissue is not fluidized, the interaction would be detected with external means, for example, by a difference frequency only detected when two shear waves interact nonlinearly, with their disappearance correlated to tissue disruption.

Acoustic Emission: As a tissue volume is disturbed, its effect on microbubbles formation in the tissue is changed. For example, microbubbles may grow more easily and have a different lifetime and collapse changing characteristics in intact versus disturbed tissue. Microbubbles may also move and interact after mechanical deformation producing larger bubbles or cooperative interaction among bubbles, all of which can result in changes in acoustic emission. These emissions can be heard during treatment and they can change during treatment. Analysis of these changes, and their correlation to therapeutic efficacy, enables monitoring of the progress of therapy.

Electrical Impedance Tomography: An impedance map of a therapy site can be produced based upon the spatial electrical characteristics throughout the therapy site. Imaging of the conductivity or permittivity of the therapy site of a patient can be inferred from taking skin surface electrical measurements. Conducting electrodes are attached to a patient's skin and small alternating currents are applied to some or all of the electrodes. One or more known currents are injected into the surface and the voltage is measured at a number of points using the electrodes. The process can be repeated for different configurations of applied current. The resolution of the resultant image can be adjusted by changing the number of electrodes employed. A measure of the electrical properties of the therapy site within the skin surface can be obtained from the impedance map, and changes in tissue in the histoplasty process can be monitored using this process.

Second-Harmonic Imaging Microscopy: The cell and tissue structure and function can be visualized using a microscope imaging contrast mechanism such as second-harmonic imaging microscopy (SHIM) which obtains contrasts from variations in a tissue specimen's ability to generate second-harmonic light from incident light. An intense laser light passes through the tissue (having a non-centrosymmetric molecular structure) which is either inherent or induced externally, for example, by an electric field. By using near infrared wavelengths for the incident light, SHIM can construct three-dimensional images of specimens by imaging deeper into thick tissues. A comparison of images can give insight about the changes in the microstructure of the tissue or collagen structure that can be monitored using this process, for example, as described in Example 2 below.

Histoplasty Parameter Adjustments:

In some embodiments of the present teachings, opportunities exist to adjust or customize the histoplasty process for particular applications. By changing various parameters, intermediate-intensity pulses can be delivered, therapy intensity varied, and changes in treatment pulses can be realized. The aforementioned feedback and monitoring methods readily allow these directed parameter adjustments and the effects thereof to be observed during the histoplasty process, in real time, and/or permit therapy progress measurement in stages, where therapy can be reinitiated as desired or as necessary.

Intensity thresholds of treatment pulses can also be varied as needed. The feedback and monitoring methods of the present disclosure allow changes in intensity to be observed in real time or in stages as desired. Changes in intensity can identify and tune intensity thresholds for ultrasound induced mechanical deformation in order to achieve localized and discrete soft tissue disruption without cavitation.

Additional parameter adjustments can affect the structure of tissue lesions produced by the histoplasty process. For example, adjustment of specific acoustic parameters, such as pulse sequence repetition frequency (PRF) and sustaining pulse amplitude, can result in marked effects on the physical characteristics of resulting tissue damage. Sensitivity of disrupted tissue production to acoustic input parameters can provide a means by which to exert control over the degree to which the mechanical effects of localized ultrasound are responsible for lesion formation.

Therapeutic Applications:

In some embodiments, the pulsed non-cavitational ultrasound methods of the present teachings permit various therapeutic procedures, including tissue deformation via controlled mechanical movement of soft tissue, cell or tissue remodeling, or drug delivery and activation, to be accomplished either wholly from means external to the body, or with minimal dependence on procedures no more invasive than current endoscopic techniques. Being noninvasive, the cost advantages, both in hospital stay and in surgical preparation time, are readily apparent. In addition, the reduction or absence of cosmetic disfigurement and risk of infection are both significant advantages. While this noninvasive property is shared with other ultrasound based delivery methods, histoplasty according to the present teachings has several potential advantages over current approaches.

In some embodiments, therapies based on the present teachings can include the following features: ability to use a low ultrasound frequency, which will not heat intervening tissue; ability to use a frequency low enough to propagate through some bone interfaces such a ribs or skull; and ability to use a frequency low enough to make phased array element sizes larger thus significantly reducing array and driving system costs.

In some embodiments, therapies based on the present teachings can include the following features: ability to use a very short pulse duration (e.g., a few microseconds in length) to exert increased oscillatory forces and shear stress on the tissue; ability to use a very short pulse duration to increase the wave momentum applied to the target tissue; and ability to use a very short pulse duration (along with the high pulse repetition frequency) to maintain low constant duty cycle (e.g., ≤1% or ≤2%) and minimize heating effects on the tissue.

In some embodiments, therapies based on the present teachings can include the following features: ability to use a high pulse repetition frequency (PRF) to exert increased pressure on the target tissue; and ability to use a high pulse repetition frequency (PRF) (along with the short pulse duration) to maintain low constant duty cycle (e.g., ≤1% or ≤2%) and minimize heating effects on the tissue.

In some embodiments, therapies based on the present teachings can include the following features: ability to use a low constant duty cycle (e.g., ≤1% or ≤2%) to minimize heating effects (thermal damage) on the target tissue; ability to use a low constant duty cycle (e.g., ≤1% or ≤2%), which will not heat intervening tissue; and ability to use a low constant duty cycle (e.g., ≤1% or ≤2%) to minimize thermal damage to adjacent tissue structures by heat diffusion.

In some embodiments, therapies based on the present teachings can include the following features: ability to use an intermediate pulse intensity (e.g., P−<15 MPa; P+<60 MPa) to mechanically deform the tissue without cavitation and without tissue ablation; and ability to use an intermediate pulse intensity (e.g., P−<15 MPa; P+<60 MPa) to exert oscillatory forces and shear stress on the tissue; ability to use an intermediate pulse pressure (e.g., P−<15 MPa; P+<60 MPa) to mechanically deform the tissue without cavitation and without tissue ablation; and ability to use an intermediate pulse pressure (e.g., P−<15 MPa; P+<60 MPa) to exert oscillatory forces and shear stress on the tissue.

Other various embodiments of the present disclosure can include aspects of drug delivery and drug activation using pulsed non-thermal, non-cavitational ultrasound therapy. For example, methods of the present disclosure can be used to temporally disrupt membranes to permit therapeutic agents to cross one or more membranes and reach their targets. Other embodiments can include using the histoplasty process to activate ultrasonically sensitive compounds that either become active therapeutic compounds themselves, or release active therapeutic compounds at the therapy site.

Other various embodiments of the present disclosure can include aspects of various tissue disruption applications, such as tumor disruption, cancer-associated fibroblasts (CAFs) disruption, collagen fiber disruption in, e.g., breast tissue, liver tissue, pancreas tissue, brain tissue, and the methods disclosed herein can be used in applications where tissue is deformed (i.e., remodeled, rearranged, or disrupted) in cancer treatments.

The following non-limiting examples illustrate the compositions, methods, and applications of the present teachings.

### EXAMPLES

### Example 1: Tissue Disruption Using Non-Cavitational Focused Ultrasound

The experimental apparatus 100 for ultrasound exposure and acoustic backscatter acquisition is as given in FIG. 4.

Ultrasound Transducers: The 788-kHz focused single element therapy transducer 102 (f number=1, Etalon Inc., Lebanon Ind. USA) is employed to create tissue deformation. The 5-MHz monitoring transducer 104 (Valpey Fisher Corporation, Hopkinton, Mass. USA) is mounted in the center inner hole of the 788-kHz therapy transducer 102. Acoustic backscatter waveforms are recorded using a digital oscilloscope 120 (Model 9354™, LeCroy, Chestnut Ridge, N.Y. USA).

Experimental Design: Ultrasound pulses are delivered by the 788 kHz therapy transducer 102. Acoustic backscatter from the therapy pulse at 788 kHz is received by a 5 MHz monitoring transducer 104. Acoustic backscatter waveforms are recorded using the digital oscilloscope 120.

Ultrasound pulses are administered by the 788 kHz therapy transducer 102 below the pulse peak pressure threshold to prevent cavitation within the target tissue 108. At very low duty cycles, very large peak negative pressure (P−) is required to generate spontaneous inertial cavitation. For example, single ultrasound pulses of 25 cycle length causes cavitation at the tip of a glass fiber at pressure levels in excess of about 15 MPa peak negative pressure (P−).

Therefore, the peak negative pressure (P−) is administered below the cavitation threshold, e.g., less than 15 MPa peak negative pressure (P−) and less than 10 MPa peak negative pressure (P−) and about 10 MPa peak negative pressure (P−) and about 7 MPa peak negative pressure (P−). In some embodiments, there is no requirement for the peak positive pressure (P+). In some embodiments, the peak positive pressure (P+) is less than 60 MPa and less than 50 MPa. The spatial peak pulse average intensity (I_{SPPA}) is administered at <30 kW/cm² and spatial peak time average intensity (I_{SPTA}) is administered at <0.5 W/cm².

The transducer frequency is administered by the 788 kHz therapy transducer 102 in the range of 250 kHz to 3 MHz. The pulse is comprised of 1 to 200 cycles and the pulse duration ranges from 0.5 microseconds to 20 milliseconds. The duty cycle is less than or equal to 2% or less than or equal to 1%. The peak repetition frequency (PRF) is in the range of 1 Hz to 1000 Hz. The number of pulses is in the range of 50 to 2000 pulses.

Transducer parameters may differ from histotripsy parameters with respect to at least one of the peak negative pressure (P−), peak positive pressure (P+), spatial peak pulse average intensity (I_{SPPA}), and spatial peak time average intensity (I_{SPTA}) being at lower values, as provided in FIG. 6, to prevent the formation of cavitation nuclei.

Spatial-peak pulse-average intensity (I_{SPPA}) as defined by the AIUM [AIUM, Acoustic Output Measurement Standard for Diagnostic Ultrasound Equipment, UD2-98: AIUM/NEMA, 1998.] and is often used to represent the amplitude of acoustic pulses. However, the amplitudes of the therapy pulses employed are comparable to lithotripter pulses and are highly non-linear. The broad frequency content of the highly nonlinear pressure waveforms generated may conflict with other assumptions commonly used in intensity estimations of more linear acoustic fields. Therefore, the peak negative pressure (P−) and the peak positive pressure (P+) are used as a metric for the amplitude of the acoustic therapy pulses.

### Example 2: Ultrasound Tissue Disruption of Breast Cancer Tissue

Background: Fibrotic extracellular matrix (ECM) directs cell behavior towards tumor progression. Specifically, biophysical and biochemical cues from the fibrotic ECM regulate the pathophysiology of tumor cell functions, including invasion, epithelial to mesenchymal transition, increased circulating tumor cells and metastatic outgrowth. These are all processes of disease progression that require tumor cell evasion from immune surveillance. The fibrotic ECM also activates mechano-signaling in stromal cell populations, such as carcinoma-associated fibroblasts, in primary and metastatic tumors leading to pro-tumor immune infiltration, and immunosuppressive cytokine signaling. Dense tumor ECM also creates a physical barrier that excludes T-lymphocytic cells, drives tumor hypoxia and reduces drug availability within the tumor microenvironment, all of which diminish the efficacy of immunotherapies.

The excess deposition of fibrotic ECM is prognostic of poor patient outcomes in breast cancer and other solid tumors. The ECM, which is enriched in collagen, presents a biophysical barrier limiting drug diffusion in both primary and metastatic tumors and further, drives immune evasion and tumor progression through distinct signaling pathways.

Current anti-fibrotic therapies directly targeting the ECM are limited by the short half-life of matrix proteases and the lack of specificity in delivery; likewise, therapeutic approaches targeting cancer-associated fibroblasts (CAFs) with antibody drug conjugates or the inhibition of upstream profibrotic signaling pathways are hindered by the lack of specific markers expressed on all CAFs.

In breast cancer, the deposition of collagen and fibronectin, along with other ECM proteins, are well known to surround mammary tumors. Collagen is highly abundant in primary and metastatic breast cancer and has a well-known role in hindering intratumoral penetration of therapeutics. ECM stiffness and fiber organization enhance overall breast cancer progression and are associated with poor patient outcomes. Moreover, dense fibrotic stroma within primary and metastatic tumors excludes T-lymphocytes and increases pro-tumor inflammation, demonstrating a link to immunosuppression.

As neoadjuvant therapies for triple negative breast cancer and Her2+ tumors emerge as standard of care, the successful delivery of chemotherapeutic and immunotherapeutic agents through dense tumor stroma remains a significant limitation and ongoing treatment challenge and highlights the transformative potential for a technique like histoplasty in the oncologic care of breast cancer and other solid tumors.

Histoplasty can effectively ablate small collagen fibers, thereby disrupting collagen organization and fiber alignment resulting in increased intratumoral penetration of therapeutics.

Tissue Sample: Experiments are conducted on 4T1 breast cancer tissue samples (i.e., target tissue 108).

Therapy: The experimental apparatus 100 for ultrasound exposure and acoustic backscatter acquisition is as given in FIG. 4.

The therapeutic transducer 102 is placed close to the 4T1 breast cancer tissue xenograft sample 108. The transducer 102 is coupled to a computer control device 112.

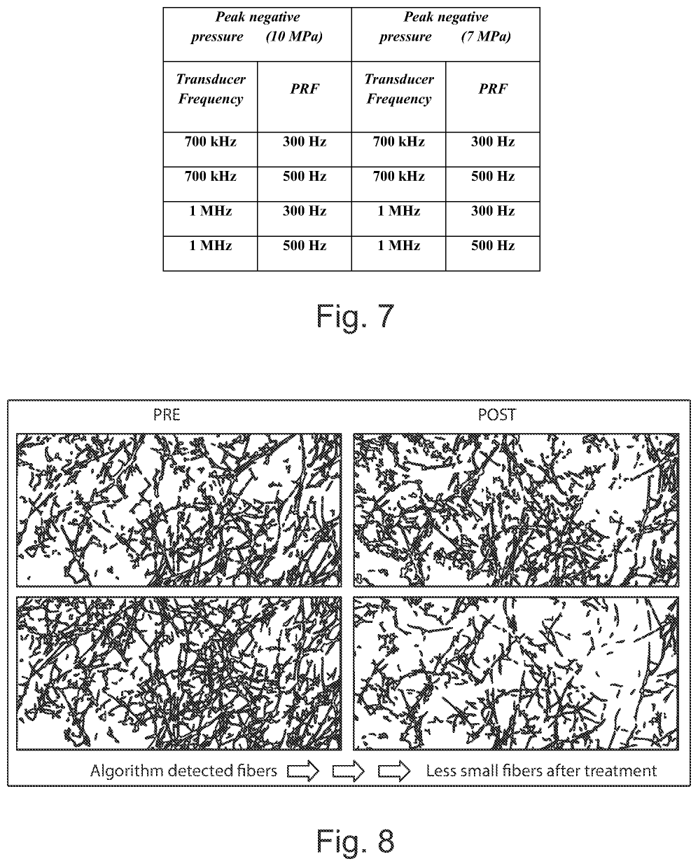

High intensity pulsed ultrasound is used to disrupt 4T1 breast cancer tissue without cavitation. The peak negative pressure (e.g., about 10 MPa and about 7 MPa) is set to prevent cavitation and with varying transducer frequency (e.g., about 700 kHz and about 1 MHz) and pulse repetition frequency (PRF) (e.g., 300 about Hz and about 500 Hz) in the various combinations listed in the table of FIG. 7. Transducer parameters are set to prevent the formation of cavitation nuclei and to maximize tissue disruption.

Results: The results are visualized using second harmonic imaging microscopy (SHIM) which presents an intense laser light passing through the tissue having a non-centrosymmetric molecular structure.

FIG. 8 graphically depicts pre-treatment and post-treatment second harmonic generation imaging of a 4T1 breast cancer tissue xenograft and demonstrates the decrease in number and size of collagen fibers post histoplasty.

This example illustrates that high intensity ultrasound can be used to mechanically disrupt collagen fibers in breast cancer tissue to a very fine degree through mechanical effects. Disrupted collagen fibers can be easily detected with standard SHIM as a substantial variation exists in the disrupted fiber's ability to generate second-harmonic light from the incident light. SHIM may be useful as direct feedback for ultrasound therapy.

### Example 3: Ultrasound Tissue Disruption in Murine Breast Carcinoma Model

Sample: 4T1 triple negative mammary carcinoma (TNBC) cells are injected into the 4th mammary fat pad of female BALB/c mice. Tumors are then allowed to grow until established (˜200 mm³). After 3 weeks, mice are euthanized and tumors immediately isolated.

Therapy: The experimental apparatus 100 for ultrasound exposure and acoustic backscatter acquisition is as given in FIG. 4.

Tumor samples 108 are imaged with second harmonic generation (SHG) imaging, treated with histoplasty, and then immediately re-imaged with SHG imaging.

Under continuous isoflurane anesthesia, mice underwent in vivo FUS treatment using a custom eight-element, small animal FUS transducer 102. The geometric focus of this transducer 102 is approximately 1-mm, 1-mm, and 3-mm in the transverse, axial and elevational directions, respectively. A custom high-voltage pulser that generates short therapy pulses<2 cycles is used to drive the transducer 102, while being controlled by a field-programmable gate array (FPGA) board (Altera DEO-Nano Terasic Technology, Dover, DE, USA) programmed for FUS therapy pulsing and powered by a high-voltage DC power supply (GENH750 W, TDK-Lambda). FUS treatments are performed on a warmed stereotactic stage coupled to a computer-guided positioning system 106 on a 3-D axis with 0.05-mm motor resolution to permit precise targeting of tumors.

Results: Referring to FIG. 8, SHG imaging of the treated tumor demonstrates significant loss of small collagen fibers with gross preservation of global tissue architecture. These results suggest histoplasty treatment can selectively decrease collagen density in the stromal matrix of breast cancer.

### Example 4: Increased Intratumoral Diffusion, Penetration, and Residence Time of Therapeutics in Murine Breast Carcinoma Model

Sample: Female BALB/c mice are inoculated with 4T1 cells to generate tumors in the mammary fat pad 108 and are allowed to grow for 2 weeks.

Therapy: The experimental apparatus 100 for ultrasound exposure and acoustic backscatter acquisition is as given in FIG. 4.

Following engraftment of 4T1 tumors, mice are injected with liposomal doxorubicin (Doxil; 2 mg/kg) and intratumoral fluorescent intensity is measured 30 min following injection (IVIS, Ex: 500 nm; Em: 620 nm). Tumoral fluorescence is calculated and normalized (number of incident photons/s/pixel). Tumors are imaged immediately following and 24-hours and 48-hours following histoplasty treatment.

Under continuous isoflurane anesthesia, mice underwent in vivo FUS treatment using a custom eight-element, small animal FUS transducer 102. The geometric focus of this transducer 102 is approximately 1-mm, 1-mm, and 3-mm in the transverse, axial and elevational directions, respectively. A custom high-voltage pulser that generates short therapy pulses<2 cycles is used to drive the transducer 102, while being controlled by a field-programmable gate array (FPGA) board (Altera DEO-Nano Terasic Technology, Dover, DE, USA) programmed for FUS therapy pulsing and powered by a high-voltage DC power supply (GENH750 W, TDK-Lambda). FUS treatments are performed on a warmed stereotactic stage coupled to a computer-guided positioning system 106 on a 3-D axis with 0.05-mm motor resolution to permit precise targeting of tumors.

Results: Sham (left flank) or histoplasty treated (right flank) tumors are subsequently imaged 24- and 48-hours following treatment.

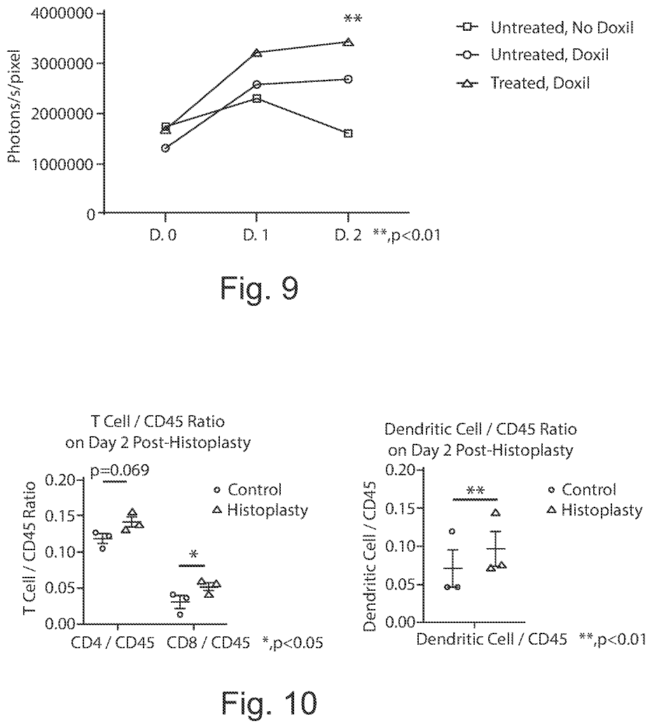

Referring to FIG. 9, histoplasty treated tumors demonstrated a statistically significant increase in intratumoral diffusion, penetration, and residence time of Doxil when compared to sham treated and untreated mice supporting the hypothesis that ablation of small collagen fibers with histoplasty alters the stromal matrix to permit increased perfusion and residence time of therapeutics like Doxil.

### Example 5: Tumor Microenvironment Favorable to Immunotherapy in Murine Breast Carcinoma Model

Sample: Female BALB/c mice are inoculated with 4T1 cells to generate unilateral flank tumors and tumors are allowed to grow for 2 weeks.

Therapy: The experimental apparatus 100 for ultrasound exposure and acoustic backscatter acquisition is as given in FIG. 4.

Tumors are then treated with histoplasty and two days following treatment, tumors are harvested for flow cytometry analysis.

Results: Referring to FIG. 10, when compared to untreated tumors, histoplasty treated tumors demonstrate increased numbers of CD8+ T-cells (left panel) and dendritic cells (right panel) as well as trending towards decreased MDSCs and Tregs, which together suggest the presence of a more favorable tumor microenvironment with decreased immunosuppression.

### Example 6: Optimize Histoplasty Parameters for Controlled and Selective Degradation of Small Collagen Fibers in Murine Breast Carcinoma Model

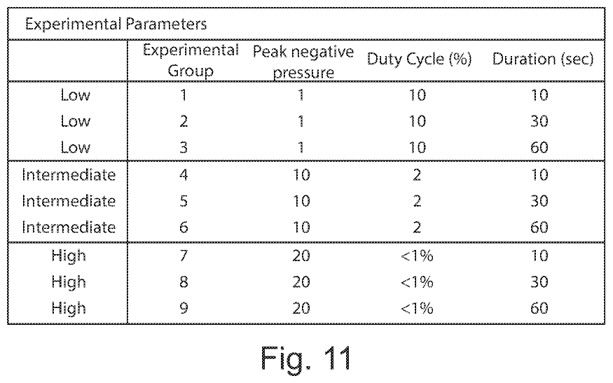

Sample: Bilateral flank 4T1 mammary tumors are generated in 8-week-old female BALB/c mice (ATCC, Manassas, VA). Once tumors are established (˜200 mm³), animals are randomized to an experimental treatment group (FIG. 11).

Therapy: The experimental apparatus 100 for ultrasound exposure and acoustic backscatter acquisition is as given in FIG. 4.

Each flank tumor in the animal is then treated with histoplasty per experimental group (n=8 per group; ntotal=72 animals) to test the effect of a range of acoustic parameters (peak negative pressure, duty cycle, duration) on the collagen density and morphology. 24-hours following histoplasty treatment, animals are euthanized, and tumors harvested.

Analysis of matrix architecture: Changes in collagen alignment between treatment groups are examined. Mammary tumors are imaged using multiphoton laser-scanning microscopy to visualize fibrillar collagen by second harmonic generation imaging (SHG). Collagen deposition and fibril orientation are analyzed using automated fiber detection software (CT FIRE and CurveAlign; loci.wisc.edu/software) and quantitatively calculated fiber density, fiber angle, fiber length, fiber straightness, and fiber width.

Statistical modeling and data analysis: To determine optimal sonication parameters for degradation of collagen fibers in the ECM, collagen density (as measured from SHG imaging) is regressed against the FUS experimental parameters (peak negative pressure, duty cycle, and exposure duration). As groups of animals undergo FUS treatment in cohorts, ‘cohort’ is included as a categorical covariate. If any relationship between collagen density and a variable are seen to be nonlinear (as evidenced by a scatterplot), higher order polynomial effects are modeled; a fit to a non-linear regression model may also be pursued; however, this is less favored due to the absence of a specified function.

Additional regression models regressing fiber angle, length, straightness, and width again FUS parameters are performed. The construction of a multiple regression models also allow for the testing and identification of important variables to the overall regression model as evidenced by the standard error of each coefficient (e.g., high standard error, low overall contribution to effect), which can inform iterative parameter optimization in planned subsequent rounds of testing. Following regression, the sign of the calculated coefficients is also expected to inform the direction to which each parameter can be further optimized (positive sign, smallest possible value; negative sign, largest possible value as a minimum in collagen density is the desired outcome).

It is understood that histoplasty can disrupt small collagen fibers from the stromal matrix of mammary carcinomas. Histoplasty parameters (peak negative pressure, duty cycle, and exposure duration) can be fine-tuned to optimally decrease collagen density in 4T1 tumors.

### Example 7: Optimize Histoplasty Parameters on Intratumoral Penetration of Therapeutics in Murine Breast Carcinoma Model

Sample: Bilateral flank 4T1 breast tumors are generated in 8-week-old female BALB/c mice (ATCC, Manassas, VA). Once tumors are established (˜200 mm³), animals are again randomized to an experimental treatment group as shown in FIG. 11.

Analysis of tumoral penetration of therapeutics: The experimental apparatus 100 for ultrasound exposure and acoustic backscatter acquisition is as given in FIG. 4.

Mice are injected with liposomal doxorubicin (Doxil; 2 mg/kg) and intratumoral fluorescent intensity is measured 30 min following injection (IVIS, Ex: 500 nm; Em: 620 nm). Tumoral fluorescence is calculated and normalized (number of incident photons/s/pixel). Tumors are subsequently imaged at 24-hours and 48-hours following histoplasty treatment.

Statistical modeling and data analysis: To determine the effect of histoplasty on intratumoral penetration of therapeutics, tumor fluorescence (normalized to imaged surface area) are regressed against the FUS experimental parameters (peak negative pressure, duty cycle, and exposure duration). As groups of animals undergo FUS treatment in cohorts, ‘cohort’ is included as a categorical covariate. If any relationship between collagen density and a variable are seen to be non-linear (as evidenced by a scatterplot), higher order polynomial effects are modeled; a fit to a non-linear regression model may also be pursued; however, this is less favored due to the absence of a specified function.

Additional regression models regressing fiber angle, length, straightness, and width again FUS parameters is performed. The construction of a multiple regression models also allowed for the testing and identification of important variables to the overall regression model as evidenced by the standard error of each coefficient (e.g., high standard error, low overall contribution to effect), which can inform iterative parameter optimization. A correlation coefficient is determined between tumor fluorescence intensity and collagen density to determine the potential association between collagen density and tumoral penetration of therapeutics.

It is understood that histoplasty can increase intratumoral perfusion of therapeutics. There is a correlation between collagen density and the degree of intratumoral perfusion. Histoplasty parameters can be fine-tuned to increase therapeutic penetration into the tumor.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “an electronic computer” and “a processor” or “the microprocessor” and “the processor,” can be understood to include one or more of these devices that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

References to “a processor” should be understood to include electronic computers, microprocessors, microcontrollers, FPGA devices, ASIC devices and similar programmable or program defined electronic circuits and collections of such devices that can communicate in a stand-alone and/or a distributed environment(s), and can thus be configured to communicate via wired or wireless communications with other processors. Furthermore, references to memory, unless otherwise specified, can include one or more processor-readable and accessible memory elements and/or components that can be internal to the processor or external to the processor and accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What I claim is:

1. A method for controlled mechanical deformation of soft tissue having a cavitation threshold initiating a bubble cloud, comprising:
outputting a treatment ultrasound pulse sequence at a treatment portion of the soft tissue without initiation of a bubble cloud in said treatment portion of the soft tissue in response to the treatment ultrasound pulse sequence;
wherein the treatment ultrasound pulse sequence is at a pulse intensity that is less than the cavitation threshold and at least partially deforms the soft tissue in the treatment portion without initiating a bubble cloud.

2. The method of claim 1 wherein a peak negative pressure of the treatment ultrasound pulse sequence being less than or equal to 20 MPa.

3. The method of claim 2 wherein a peak negative pressure of the treatment ultrasound pulse sequence being less than or equal to 15 MPa.

4. The method of claim 1 wherein a peak positive pressure of the treatment ultrasound pulse sequence being less than or equal to 60 MPa.

5. The method of claim 1 wherein a frequency of the treatment ultrasound pulse sequence is between 500 kHz and 1 MHz.

6. The method of claim 1 wherein a pulse repetition frequency (PRF) of the treatment ultrasound pulse sequence is between 250 Hz and 750 Hz.

7. The method of claim 1 wherein a duty cycle of the treatment ultrasound pulse sequence is less than or equal to 2%.

8. The method of claim 7 wherein a duty cycle of the treatment ultrasound pulse sequence is less than or equal to 1%.

9. The method of claim 1 wherein pulse length of the treatment pulse is less than or equal to 15 μsec.

10. The method of claim 1 wherein a number of pulses in the treatment ultrasound pulse sequence is 10 to 50 pulses.

11. The method of claim 1 wherein an exposure duration of the treatment ultrasound pulse sequence is less than 60 sec.

12. The method of claim 1 wherein the treatment ultrasound pulse sequence is applied to at least one of a breast cancer tissue, liver cancer tissue, and pancreas cancer tissue.

13. The method of claim 1 further comprising the step of monitoring an amount of deformation of the soft tissue in the treatment portion.

14. The method of claim 13 further comprising the step of adjusting the treatment ultrasound pulse sequence based on the amount of deformation of the soft tissue in the treatment portion.

15. A method for controlled mechanical disruption of fibrotic extracellular matrix in a human tissue having a cavitation threshold initiating a bubble cloud in the fibrotic extracellular matrix, comprising:
outputting a treatment ultrasound pulse sequence at a treatment portion of the fibrotic extracellular matrix without initiation of a bubble cloud in said treatment portion of the fibrotic extracellular matrix in response to the treatment ultrasound pulse sequence;
wherein the treatment ultrasound pulse sequence is at a pulse intensity that is less than the cavitation threshold and at least partially deforms the fibrotic extracellular matrix without initiating a bubble cloud in the fibrotic extracellular matrix.

16. The method of claim 15 wherein the fibrotic extracellular matrix is of at least one of a breast cancer tissue, liver cancer tissue, and pancreas cancer tissue.

17. The method of claim 15 further comprising the step of monitoring an amount of fibrotic extracellular matrix disruption and adjusting the treatment ultrasound pulse sequence based on the amount of disruption in the treatment portion.

18. The method of claim 15 further comprising the step of monitoring a collagen density in the fibrotic extracellular matrix and adjusting the treatment ultrasound pulse sequence based on the collagen density in the fibrotic extracellular matrix in the treatment portion.

19. The method of claim 15 further comprising the step of monitoring an amount of penetration of therapeutics into the human tissue and adjusting the treatment ultrasound pulse sequence based on the amount of penetration of therapeutics into the human tissue in the treatment portion.
